(19) United States
(12) Patent Application Publication
(10) Pub. No.: US 2003/0097041 A1
(43) Pub. Date: May 22, 2003

(54) Sex aid

(76) Inventors: Steven D. Ritchie, Treasure Island, FL (US); Harles David Reynard, Madera Beach, FL (US)

Correspondence Address: MICHAEL J. COLITZ, JR., 217 HARBOR VIEW LANE, LARGO, FL, 33770, US

(21) Appl. No.: 09/933,982

(22) Filed: Aug. 22, 2001

(51) Int. Cl. A61F 5/00

(52) U.S. Cl. 600/38

(57) ABSTRACT

A new and improved sexual aid device for increasing stimulation during sex acts. The device comprises an elongated, substantially unshaped body having a first end and a second end. The first end of the body has a digital massaging head formed integrally therewith. The head is sized for receipt in a user's anal cavity and for exerting pressure against prostate gland for producing stimulation thereof. The second end of the body has a penile loop formed integrally therewith. The loop is sized for encircling the penis of a user to thereby position and manipulate the device. Preferably, the body, head and loop are fabricated of a generally lubricous glass-based material containing an appreciable amount of an oxide of boron to render it lubricious and resistant to heat, chemicals and electricity. In use, the penile loop of the device is positioned around a user's penis such that the body of the device extends between the user's legs and toward the anus of the user thereby positioning the massaging head within the rectum of the user adjacent the prostate gland.

### BACKGROUND OF THE INVENTION

1. Field of the Invention

The present invention relates to a new and improved sex aid and, more particularly, pertains to increasing stimulation during sex acts.

2. Description of the Prior Art

The use of sex aids of known designs and configurations is known in the prior art. More specifically, sex aids of known designs and configurations heretofore devised and utilized for the purpose of increasing sexual stimulation through known methods and apparatuses are known to consist basically of familiar, expected, and obvious structural configurations, notwithstanding the myriad of designs encompassed by the crowded prior art which has been developed for the fulfillment of countless objectives and requirements.

The prior art discloses a large number of sex aids of known designs and configurations. By way of example, U.S. Pat. No. 5,853,362 to Jacobs discloses a glandular stimulator device and method.

In this respect, the sex aid according to the present invention substantially departs from the conventional concepts and designs of the prior art, and in doing so provides an apparatus primarily developed for the purpose of increasing stimulation during sex acts.

Therefore, it can be appreciated that there exists a continuing need for a new and improved sex aid which can be used for increasing stimulation during sex acts. In this regard, the present invention substantially fulfills this need.

### SUMMARY OF THE INVENTION

In view of the foregoing disadvantages inherent in the known types of sex aids of known designs and configurations now present in the prior art, the present invention provides a new and improved sex aid. As such, the general purpose of the present invention, which will be described subsequently in greater detail, is to provide a new and improved sex aid and methods which have all the advantages of the prior art and none of the disadvantages.

To attain this, the present invention essentially comprises a new and improved sexual aid device for increasing stimulation during sex acts. The device includes a body. The body is substantially u-shaped. The body has a first end and a second end.

The device also included a digital massaging head. The digital massaging head is formed integrally with the first end of the body. The head is sized for receipt in a user's anal cavity and for exerting pressure against prostate gland for producing stimulation thereof.

The second end of the body has a penile loop formed integrally therewith. The loop is sized for encircling the penis of a user to thereby position and manipulate the device.

Preferably, the body, head and loop are fabricated of a generally lubricous glass-based material containing an appreciable amount of an oxide of boron to render it lubricious and resistant to heat, chemicals and electricity. In use, the penile loop of the device is positioned around a user's penis such that the body of the device extends between the user's legs and toward the anus of the user thereby positioning the massaging head within the rectum of the user adjacent the prostate gland.

There has thus been outlined, rather broadly, the more important features of the invention in order that the detailed description thereof that follows may be better understood and in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an object of the present invention to provide a new and improved sex aid which has all the advantages of the prior art sex aids of known designs and configurations and none of the disadvantages.

It is another object of the present invention to provide a new and improved sex aid which may be easily and efficiently manufactured and marketed.

It is a further object of the present invention to provide a new and improved sex aid which is of a durable and reliable construction.

It is yet a further object of the present invention to provide a new and improved sex aid which may be readily sterilized for improved sanitation.

An even further object of the present invention is to provide a new and improved sex aid which is susceptible of a low cost of manufacture with regard to both materials and labor, and which accordingly is then susceptible of low prices of sale to the consuming public, thereby making such a sex aid economically available to the buying public.

Even still another object of the present invention is to increase stimulation during sex acts.

Lastly, it is an object of the present invention to provide a sexual aid comprising an elongated, substantially unshaped body having a first end and a second end. The first end of the body has a digital massaging head formed integrally therewith. The head is sized for receipt in a user's anal cavity and for exerting pressure against prostate gland for producing stimulation thereof. The second end of the body has a penile loop formed integrally therewith. The loop is sized for encircling the penis of a user to thereby position and manipulate the device. Preferably, the body, head and loop are fabricated of a generally lubricous glass-based material containing an appreciable amount of an oxide of boron to render it lubricious and resistant to heat, chemicals and electricity.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is perspective view of the sex aid constructed in accordance with the principles of the present invention.

FIG. 2 is an side elevational view taken along line 2-2 of FIG. 1.

FIG. 3 is a end view taken along line 3-3 of FIG. 2.

FIG. 4 is a top plan view taken along line 4-4 of FIG. 1.

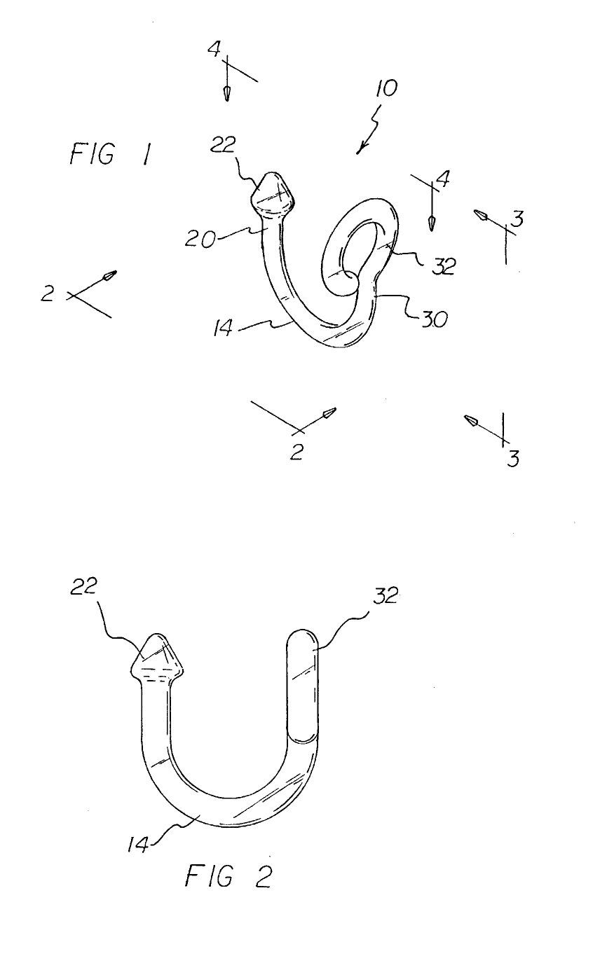

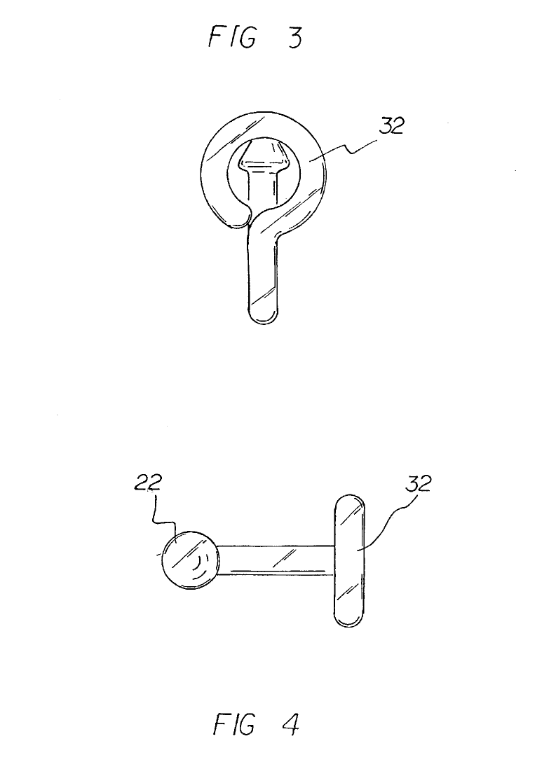

The same reference numerals refer to the same parts throughout the various Figures.

### DESCRIPTION OF THE PREFERRED EMBODIMENT

With reference now to the drawings, and in particular to FIG. 1 thereof, the preferred embodiment of the new and improved sex aid embodying the principles and concepts of the present invention and generally designated by the reference numeral 10 will be described.

The present invention, the new and improved sex aid, is a device 10 comprised of a plurality of components. Such components, in their broadest context, include a body, a first end, and a second end. Each of the individual components is specifically configured and correlated one with respect to the other so as to attain the desired objectives.

The first component of the new and improved sexual aid device 10 for increasing stimulation during sex acts is a body 14. The body is formed of an elongated, substantially unshaped body. The body has a first end 20 and a second end 30. The body is sized to extend from the base of a user's phallic region to a position adjacent the user's prostate gland. The size of the body can be varied as needed depending on the anatomical requirements of a user.

The second component of the device is a digital massaging head 22. The digital massaging head is formed integrally with the first end of the unshaped body. The head sized for receipt in a user's anal cavity and for exerting pressure against prostate gland. The size of the head can be varied as needed depending on the anatomical requirements of a user.

The third component of the device is a penile loop 32. The penile loop is formed integrally with the second end of the body. The loop sized for encircling the penis of a user for positioning and manipulating the device. The size of the loop can be varied as needed depending on the anatomical requirements of a user.

All components of the sexual aid devive, the body, digital massaging head and penile loop, are fabricated of a generally lubricous glass-based material. This glass-based material contains an appreciable amount of an oxide of boron to render it lubricious and resistant to heat, chemicals, electricity and bacterial absorption.

The present invention is a stimulation device designed for prostate gland stimulation. The embodiment has a unique grouping of a massaging head, body and penile encircling loop for stimulation, handling and motivation of the device.

In use, the penile loop of the device is positioned around a user's penis such that the body of the device extends between the user's legs and toward the anus of the user thereby positioning the massaging head within the rectum of the user adjacent the user's prostate gland. During intercourse, thrusting by a user imparts a rocking motion to the device as a result of the penile loop contacting the penetrated partner's body and also thereby directly stimulating the penetrated partner. The rocking motion imparted to the device causes the massaging head to contact and stimulate the user's prostate gland.

Each of the embodiments of the present invention are hand-crafted from solid glass-based shafts. As such, the devices will stay slick for a long time without reapplying extra lubrication or rewetting. In addition, the devices will not hold odor. Each device is seamless, and created individually, one at a time. The devices are inspected carefully to insure perfect smoothness. The devices may be sterilized, washed in soap and water, wiped with alcohol or autoclaved. The devices may be used with water and oil-based lubricants. Unlike conventional water-based lubricants used to protect latex condoms which become sticky when used on rubber-like devices, the present invention provides smooth contact with rectal tissues. This arrangement of components and materials renders the present invention very suitable for mass production.

As to the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed as being new and desired to be protected by Letters Patent of the United States is as follows:

1. A new and improved sexual aid device for increasing stimulation during sex acts comprising, in combination:
an elongated, substantially u-shaped body having a first end and a second end;
a digital massaging head formed integrally with the first end of the u-shaped body, the head sized for receipt in a user's anal cavity and for exerting pressure against prostate gland; and
a penile loop formed integrally with the second end of the body, the loop sized for encircling the penis of a user for positioning and manipulating the device.

2. The sex aid as set forth in claim 1 wherein the body, head and loop are fabricated of a generally lubricous glass-based material containing an appreciable amount of an oxide of boron to render it lubricious and resistant to heat, chemicals and electricity.

3. A new and improved sexual aid device for increasing stimulation during sex acts comprising, in combination:
a substantially u-shaped body having a first end and a second end;
a digital massaging head formed integrally with the first end of the u-shaped body; and
a penile loop formed integrally with the second end of the body.

4. The sex aid as set forth in claim 3 wherein the body, head and loop are fabricated of a generally lubricous glass-based material containing an appreciable amount of an oxide of boron to render it lubricious and resistant to heat, chemicals and electricity.

5. A new and improved sexual aid device for increasing stimulation during sex acts comprising, in combination:
a u-shaped body having a first end and a second end;
a prostate massaging head formed integrally with the first end of the u-shaped body; and
a penile loop formed integrally with the second end of the body.
